(19) United States
(12) Patent Application Publication
(10) Pub. No.: US 2022/0287789 A1
(43) Pub. Date: Sep. 15, 2022

(54) SURGICAL DRAPE OR SHEATH FOR A MEDICAL VACUUM DEVICE AND METHODS OF USE

(71) Applicant: David Louis Kirschman, Dayton, OH (US)

(72) Inventor: David Louis Kirschman, Dayton, OH (US)

(73) Assignee: Aerobiotix, Inc., Miamisburg, OH (US)

(21) Appl. No.: 17/560,443

(22) Filed: Dec. 23, 2021

Related U.S. Application Data
(60) Provisional application No. 63/158,964, filed on Mar. 10, 2021.
(60) Provisional application No. 63/216,278, filed on Jun. 29, 2021.

(51) Int. Cl. A61B 46/10; B01D 46/02

(52) U.S. Cl. CPC A61B 46/10; B01D 46/02; B01D 2273/28; B01D 2275/201; B01D 2279/00

(57) ABSTRACT

A surgical drape or sheath system, apparatus and method is shown for reducing or eliminating contamination. The surgical drape or sheath system, apparatus and method comprise at least one body that is mounted on or in the vacuum tube while a generally tubular drape or sheath is draped or positioned over at least a portion of the vacuum tube so that it can be grasped by a user. The surgical drape or sheath system, apparatus and method may be disposable or may be provided with reusable components or components that can be replaced.

### CROSS-REFERENCE TO RELATED APPLICATION

The present application claims priority to provisional U.S. Application Ser. No. 63/158,964 filed Mar. 10, 2021, and to provisional U.S. Application Ser. No. 63/216,278 filed Jun. 29, 2021, to which Applicant claims the benefit of the filing dates of these provisional applications, both of which are incorporated herein by reference and made a part hereof.

### BACKGROUND OF THE INVENTION

### 1. Field of the Invention

This invention relates to a system and method for facilitating, preventing or reducing transfer of contamination from a vacuum device to a person, such as a nurse, practitioner or doctor, or patient, or to a surgical environment. The system and method utilize a protective drape or sheath and body that are used with or mounted on the vacuum tube such that a user can grasp the vacuum tube without transferring bacteria or contaminants residing thereon to the user, to a person or into the environment.

### 2. Description of the Related Art

Surgical procedures can generate significant levels of localized air contamination with chemical and biological agents. These agents can include smoke arising from electrocauterization or bacteria arising from aerosolized infectious agents. Several devices are prevalent in the prior art, notably the device described in U.S. Pat. No. 10,702,435 which discloses a negative-pressure air device incorporating a semi-rigid vacuum tube with a nozzle that can be positioned near a surgical site or aerosol-generating procedure.

A drawback of this and similar systems is that sterilizing a large vacuum tube, which may have a diameter of 2-3 inches and length of 4-6 feet, for each procedure is impractical. Therefore, the vacuum tube itself cannot directly contact a sterile surgical site due to risk of contamination.

What is needed, therefore, is a system and method for overcoming one or more of the problems of the past and for providing a system and method for addressing contamination on a vacuum tube.

### SUMMARY OF THE INVENTION

The invention provides a device for allowing a non-sterile vacuum tube to be safely used in a sterile surgical procedure. Because of the flexible nature of the vacuum tube, such the system, method and device incorporates a flexible, impermeable surgical drape or sheath component, as well as a gas-permeable component that prevents gas to flow therethrough. This drape or sheath functions to prevent the spread of contamination between the vacuum tube and the surgical site, whilst allowing vacuum air to pass therethrough.

One object of the invention is to provide a surgical drape or sheath system for use with a vacuum tube in a medical environment.

Another object of the invention is to provide a surgical drape or sheath system and method that facilitate preventing or reducing a transfer of contaminants to a component in a surgical environment, to a person, such as a nurse, doctor, patient or the like or to the environment where the patient is located, such as a patient room or a surgical operating room.

Another object of the invention is to provide a sheath and drape system and method that comprises a body having a flexible and non-permeable drape or sheath that can be mounted on a vacuum tube that is used in a surgical environment.

Yet another object of the invention is to provide a surgical drape or sheath having a body that is mounted on a vacuum tube and that has a flexible drape and sheath component that is generally tubular and that can be draped over the vacuum tube in order to reduce or eliminate the chance of any bacteria or contamination on the vacuum tube from being transferred to a person or into the local environment.

Another object of the invention is to provide a sheath and drape system and method that comprises a body that is mounted on an end of the vacuum tube.

Still another object of the invention is to provide a sheath and drape system that comprises a body and a flexible sheath and drape wherein the body is mounted inside the vacuum tube and completely concealed by the vacuum tube, while the drape is draped over the outer surface of the vacuum tube.

Still another object of the invention is to provide a sheath and drape system having a body that is mounted inside the vacuum tube, while other embodiments provide means and apparatus for mounting the body on an outside surface of the vacuum tube.

Yet another object of the invention is to provide a sheath and drape system having a filter located in a nozzle end of the body, with the nozzle end and the filter having a complementary shape and in at least some embodiments being frusto-conical in cross-section.

Another object of the invention includes providing a sheath and drape system wherein the body comprises a frusto-conical shape and is matingly received in an end of the vacuum tube or nozzle which also comprises a mating frusto-conical shape.

Still another object of the invention is to provide means and apparatus for securing the body of the surgical drape or sheath system to a vacuum tube.

Another object of the invention is to provide a surgical drape or sheath system that is sterilizable and re-usable and/or is disposable, for example, after a one-time or multiple time use.

In one embodiment, the invention consists of a single-use disposable flexible tubular sheath which is placed over the vacuum tube and incorporates a concave conical air filtration means in proximity to the vacuum tube nozzle. In another embodiment, the tubular sheath incorporates an elongated sterile extension to create a sterile transition zone between the surgical site and the vacuum tube. In still another embodiment, the sheath is positioned in a U-shape such that it extends substantially into the interior of the vacuum tube, terminating in a gas-permeable region and maintaining a sterile transition zone.

In one aspect, one embodiment of the invention comprises a surgical sheath for use with a vacuum device having a vacuum tube, the surgical sheath comprising a body having an open end and a second end, the body being adapted and sized to be coupled to or in fluid communication with an end of the vacuum tube, a sheath coupled to the second end of the body and adapted to receive at least a portion of the vacuum tube; and a filter for mounting to the second end of the body and filtering air that flows therethrough, the body not permitting airflow peripheral to the filter and the sheath and the body cooperating to substantially prevent air movement between the sheath and the vacuum tube after the body is detachably mounted thereto.

In another aspect, another embodiment of the invention comprises a method for preventing contamination of a vacuum hose used in a surgical operating room, the vacuum hose comprising a distal end, the method comprising the steps of placing a substantially tubular sheath over the vacuum hose, the substantially tubular sheath comprising a filtration area having a filter; coupling the filtration end to the distal end so that air passing into the vacuum hose gets filtered by the filter.

In yet another aspect, another embodiment of the invention comprises a surgical sheath for use with a medical vacuum device having a vacuum end, the surgical sheath comprising a substantially flexible body that becomes situated around at least a portion of the medical vacuum device; a substantially rigid extension having a coupling end for detachably mounting to the vacuum end such that the substantially flexible body becomes situated around at least a portion of the medical vacuum device; a filter situated at the coupling end of the substantially rigid extension for filtering air before it enters the medical vacuum device.

In still another aspect, another embodiment of the invention comprises a method for preventing contamination of a sterile field in a surgical operating room when using a medical vacuum device having a vacuum tube, the method comprising the steps of placing a substantially flexible tubular sheath over the vacuum tube used in the surgical operating room, the vacuum tube comprising a distal end; coupling an integral sterile substantially rigid extension to an inlet end of the medical vacuum device; placing a substantially tubular sheath over at least a portion of the vacuum tube, the substantially tubular sheath comprising a filtration end having a filter; the substantially tubular sheath being adapted and sized to receive at least a portion of the vacuum tube, thereby creating a sterile transition zone over at least one of a length of the substantially rigid extension or the at least a portion of the vacuum tube.

In another aspect, another embodiment of the invention comprises a surgical drape system for use with a vacuum tube in a surgical environment, the surgical drape system comprising a first portion for coupling to an inlet end of the vacuum tube, the first portion having a first portion inlet end; a drape portion coupled to or integral with the first portion for situating about at least a portion of the vacuum tube; and the first portion having an outlet end coupled to the inlet end of the vacuum tube and the drape portion being situated about the at least a portion of the vacuum tube to facilitate preventing contamination from the vacuum tube from spreading into the surgical environment.

This invention, including all embodiments shown and described herein, could be used alone or together and/or in combination with one or more of the features covered by one or more of the following list of features:
The surgical sheath wherein the body is impermeable to air and the filter is mounted on the second end to filter air passing into the vacuum tube.
The surgical sheath wherein openings of said filter have a diameter of less than or equal to 1 mm in diameter.
The surgical sheath wherein the filter comprises a substantially convex conical shape.
The surgical sheath wherein the filter comprises a generally conical shape, the open end being sized, dimensioned and adapted to have a generally complementary shape as the filter and adapted to receive and support the filter so that it filters the air passing into the vacuum tube.
The surgical sheath wherein the body and the vacuum tube comprises a connection for providing a substantially air-tight seal between the open end and the vacuum tube.
The surgical sheath wherein the connection is reversible and comprising complementary shaping, tapering, fastening, compressing, plugging, sliding, pinning, keying, threading, rotating, magnetically attracting, hook and loop connecting, or comparable means.
The surgical sheath wherein the filter comprises open-cell foam, sieve, textile, paper, fiber or similar air filtration means; the sealing comprising a substantially air-tight border comprising heat seal, adhesive, fastener or similar joining means.
The surgical sheath wherein the surgical sheath is manufactured from impermeable flexible polymer, such as polyethylene, low-density polyethylene (LDPE) polypropylene. poly(tetrafluoroethylene) or silicones.
The method wherein the substantially tubular sheath comprises an air-impermeable body adapted to be mounted on the distal end of the vacuum hose and a non-permeable sheath portion for facilitating preventing contamination of an external surface of the vacuum hose.
The surgical sheath wherein the filter is generally conically shaped and is situated in the coupling end of the substantially flexible body.
The surgical sheath wherein the substantially rigid extension is impermeable to air and the filter is mounted on the coupling end to filter air passing into the medical vacuum device.
The surgical sheath wherein the filter the coupling end being sized, dimensioned and adapted to have a generally complementary shape as the filter and adapted to receive and support the filter so that the filter filters the air passing into the vacuum device.
The surgical sheath wherein the medical vacuum device comprises a connection for providing a substantially air-tight seal between substantially rigid extension and the vacuum device.
The surgical sheath wherein the coupling comprises a filter which is sealed to the coupling; the filter comprises open-cell foam, sieve, textile, paper, fiber or similar air filtration means; the sealing comprising a substantially air-tight border comprising heat seal, adhesive, fastener or similar joining means.
The method wherein the substantially rigid extension comprises an air-impermeable body adapted to be mounted on the vacuum end of the vacuum tube and the substantially tubular sheath comprises a non-permeable sheath portion for facilitating preventing contamination of an external surface of the vacuum tube.
The surgical drape system wherein the drape is secured to the first portion with a substantially air-tight seal defined by at least one of a weld, adhesive, elastic band, tie, press fit, strap, hook and tie, threads or the like.
The surgical drape system wherein the first portion defines an extender or spacer for separating a distance between the inlet end of the vacuum tube and the first portion inlet end for receiving contaminated air, the drape facilitating preventing the contamination from contaminating the vacuum tube.
The surgical drape system wherein the drape is coupled or secured to an end of the first portion and extends over the vacuum tube.
The surgical drape system wherein the drape is coupled or secured to an end of the first portion and extends over the vacuum tube and at least a portion of the first portion.
The surgical drape system wherein the drape and the first portion are impermeable to air; the surgical drape system having at least one filter situated in the first portion inlet end to permit contaminated air to enter the first portion and then the vacuum tube.
The surgical drape system wherein the first portion inlet end comprises a predetermined shape, the at least one filter being sized and adapted to complement a shape.
The surgical drape system wherein the predetermined shape is a frusto-conical shape.
The surgical drape system wherein the outlet end of the first portion is sized and adapted to be received in the inlet end of the vacuum tube so that the drape drapes over an outer surface of the vacuum tube to facilitate preventing contamination from transferring from the vacuum tube to the surgical environment, the outlet end being secured to the inlet end of the vacuum tube with a substantially air-tight seal.
The surgical drape system wherein the inlet end of the vacuum tube is sized and adapted to receive the outlet end of the first portion so that the drape drapes over an outer surface of the vacuum tube to facilitate preventing contamination from transferring from the vacuum tube to the surgical environment, the inlet end of the vacuum tube being secured to the outlet end with a substantially air-tight seal.
The surgical drape system wherein the outlet end is sealingly coupled to the vacuum tube so that the drape drapes over an outer surface of the vacuum tube to facilitates preventing contamination from transferring from the outer surface of the vacuum tube to the surgical environment The surgical drape system wherein the inlet end of the first portion is frusto-conical in cross section, the first portion having a complementary shape so that it fits inside the inlet end of the vacuum tube, the first portion having a filter that is also frusto-conical in shape and adapted to be housed in the inlet end of the first portion.
The surgical drape system wherein the drape is integral with or secured to an inlet end of the first portion, the outlet end of the first portion being situated in proximity to the inlet end of the vacuum tube after the first portion is mounted thereon, the drape being adapted to be situated over both the first portion and at least a portion of the vacuum tube.
The surgical drape system wherein the surgical drape system further comprises at least one of an air handler or HVAC system coupled to an inlet end of the vacuum tube for creating a negative pressure or vacuum in the vacuum tube in order to vacuum air into an inlet end of the first portion after it is mounted on the vacuum tube.

This invention, including all embodiments shown and described herein, could be used alone or together and/or in combination with one or more of the features covered by one or more of the following list of features:

These and other objects and advantages of the invention will be apparent from the following description, the accompanying drawings and the appended claims.

### BRIEF DESCRIPTION OF THE ACCOMPANYING DRAWINGS

FIG. 1A is a perspective view of an embodiment showing a body comprising a geo-impermeable surgical drape or sheath system for use on a medical vacuum device;

FIG. 1B is another view of the embodiment shown in FIG. 1A;

FIG. 1C is a cross-sectional fragmentary view of the device shown in FIG. 1A illustrating various features of the frusto-conically shaped end of a body that is adapted to receive a mating filter that is also frusto-conically shaped;

FIG. 1D is a sectional view taken along the line 1D-1D in FIG. 1A, showing various details of the embodiment of FIG. 1A;

FIG. 1E is a sectional view taken along the line 1E-1E in FIG. 1A, showing various details of the embodiment of FIG. 1A;

FIG. 2A is a view of another embodiment illustrating the surgical drape or sheath body having a frusto-conical inlet end having a similarly-shaped filter therein and an outlet end that is coupled to the vacuum tube as shown;

FIG. 2B is an cross-sectional fragmentary view of the embodiment shown in FIG. 2A;

FIG. 2C is an exploded view of the embodiment shown in FIG. 2A showing various details of this embodiment;

FIG. 2D is a sectional view taken along the line 2D-2D in FIG. 2A, showing various details of the embodiment of FIG. 2A;

FIG. 3A is a view of another embodiment showing a frusto-conically shaped body of a surgical drape or sheath system that is adapted to be received in a frusto-conically shaped funnel on an end of the vacuum tube as shown;

FIG. 3B is a cross-sectional fragmentary view of the embodiment shown in FIG. 3A;

FIG. 3C is an exploded view of the embodiment shown in FIG. 3A showing various details of this embodiment;

FIG. 3D is a sectional view taken along the line 3D-3D in FIG. 3A, showing various details of the embodiment of FIG. 3A

FIG. 4A is a view of another embodiment of the invention;

FIG. 4B is a cross-sectional fragmentary view of the embodiment shown in FIG. 4A;

FIG. 4C—4F are various views illustrating different fasteners or fastener means for coupling the surgical drape or sheath system and apparatus to a vacuum device, such as the vacuum tube;

FIG. 5A is a view of another embodiment of the invention;

FIG. 5B is a fragmentary sectional view of the embodiment shown in FIG. 5A;

FIG. 5C is a sectional view taken along the line 5C-5C in FIG. 5A, showing various details of the embodiment of FIG. 5A;

FIG. 6A is another embodiment of the invention illustrating a body of the drape or sheath system or assembly inserted completely within the vacuum tube and the tubular transparent impermeable drape or shield being situated over the vacuum tube as illustrated;

FIG. 6B is a fragmentary sectional view of the embodiment shown in FIG. 6A; and

FIG. 6C is a sectional view taken along the line 6C-6C in FIG. 6A, showing various details of the embodiment of FIG. 6A.

### DETAILED DESCRIPTION OF THE PREFERRED EMBODIMENTS

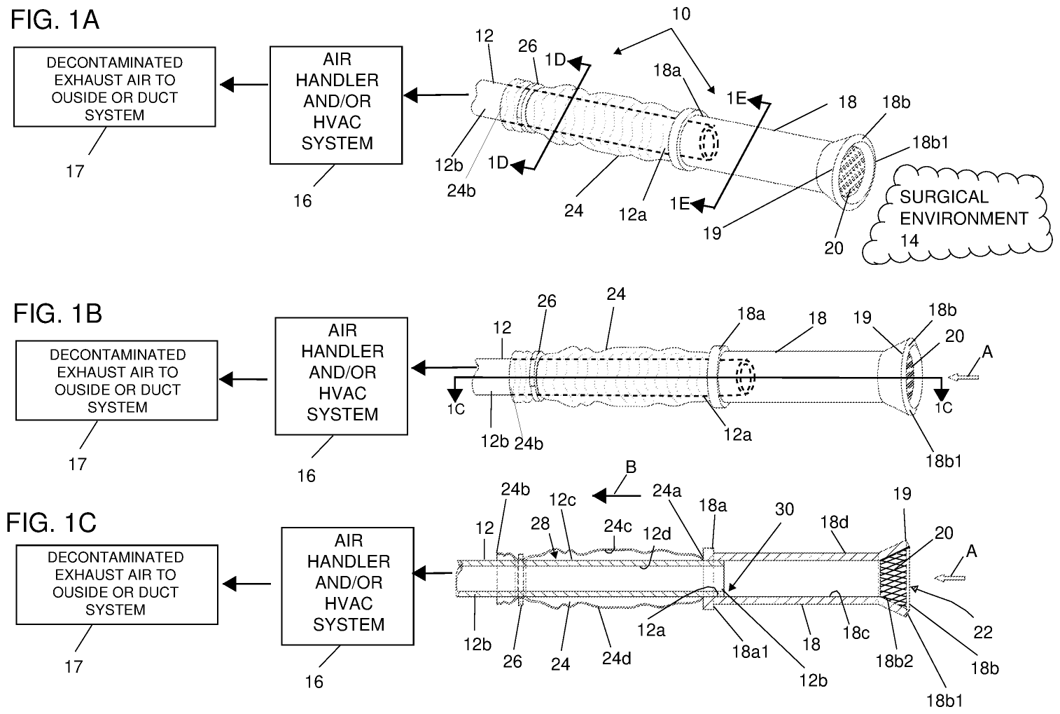

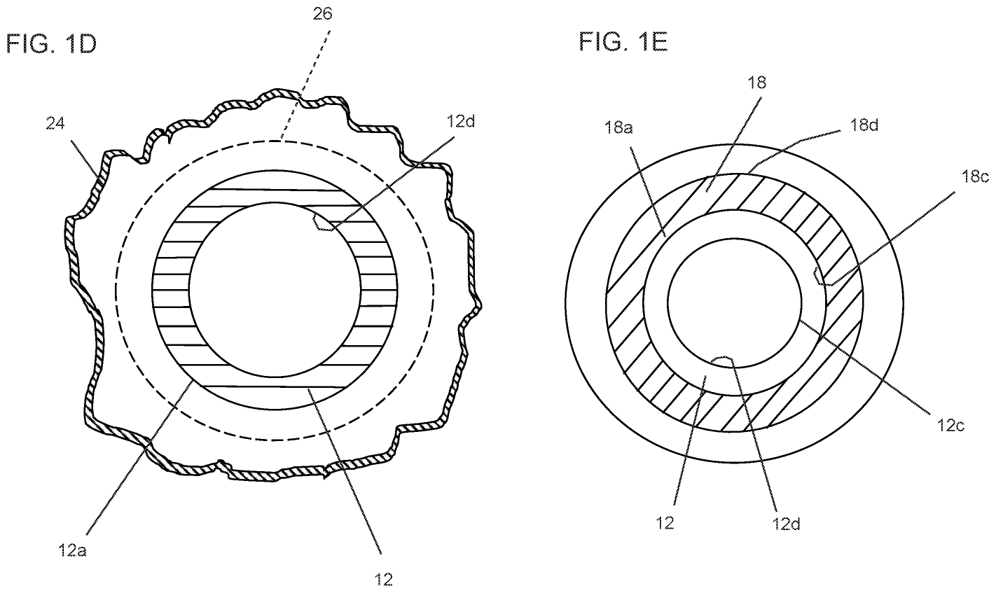

FIGS. 1A-6C illustrate several illustrative embodiments of the invention. For ease of understanding, the embodiments are grouped in separate figures (e.g., FIGS. 1A-1E, 2A-2D, 3A-3D, 4A-4F, 5A-5C and 6A-6C). Each group of figures illustrate a different embodiment. In these embodiments, the same parts are identified with the same part numbers, except that one or more prime mark(s) have been added to these like part numbers for the different embodiments. In short, the embodiments illustrate and describe a surgical drape or sheath system 10, wherein a single-use disposable flexible tubular sheath or drape apparatus 24 is placed over a vacuum hose or tube 12 in order to facilitate preventing or reducing the risk of any contaminants or bacteria on the vacuum hose or tube 12 from being transferred to a person or into a surgical environment 14. These other illustrative embodiments will now be described in detail in an order that facilitates understanding.

It should be understood that these illustrative embodiments being described provide for allowing the non-sterile or contaminated vacuum hose or tube 12 to be safely used in the sterile surgical environment 14. Accordingly, both a body 18 and the sheath or drape apparatus 24 are non-gas permeable.

In the illustration being described, the air handler or HVAC system 16 may comprise or be situated in or used with an ILLUVIA® decontamination air handler product available from Aerobiotix, Inc. of Miamisburg, Ohio. Also, it should be understood that the air handler or HVAC system 16 may comprise one or more of the features shown in the ILLUVIA® decontamination air handler product available from Aerobiotix, Inc. of Miamisburg, Ohio and the products and systems illustrated in the U.S. Pat. Nos. 9,433,693; 9,457,119; 9,764,054; 10,039,854; 10,532,122 and 10,549,007; 10,702,435; 11,052,168 and 11,123,674, all of which are assigned to the same assignee as the present application and are incorporated herein by reference and made a part hereof.

Referring now to a first embodiment in FIGS. 1A-1E, the surgical drape or sheath system 10 is shown for use with the vacuum hose or tube 12 for use in the surgical environment 14. The surgical drape or sheath system 10 comprises an air handler or HVAC system 16 that is adapted to receive contaminated air from the surgical environment 14 in order to decontaminate or treat the contaminated air and then to exhaust decontaminated air into the surgical environment 14, outside or through a duct system 17 as illustrated in FIGS. 1A-1E.

In the illustration being described, the air handler and/or HVAC system 16 receives contaminated air through the vacuum hose or tube 12, decontaminates or treats the air, and then exhausts the decontaminated air into the surgical environment 14, outside or through the duct system 17 shown in FIGS. 1A-1E. For ease of illustration, the vacuum hose or tube 12 comprises a first or inlet end 12a and a second or outlet end 12b that are integrally formed with an inner wall 12d (FIG. 1C). As shown, the second or outlet end 12b of the vacuum hose or tube 12 is coupled directly to the air handler and/or HVAC system 16 so that it can deliver the contaminated air thereto. The outlet end 12b is coupled to a sheath or drape apparatus 24, which will now be described.

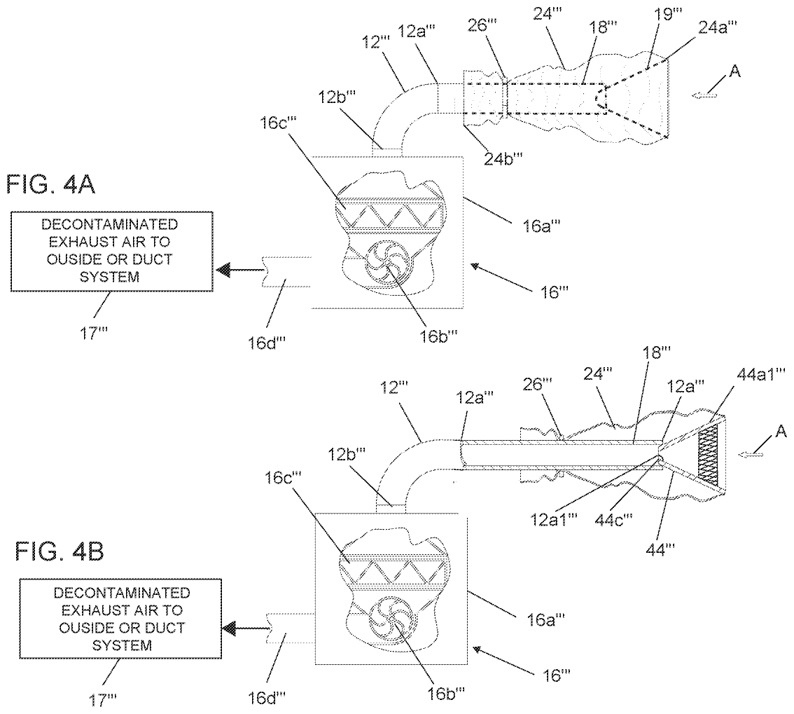

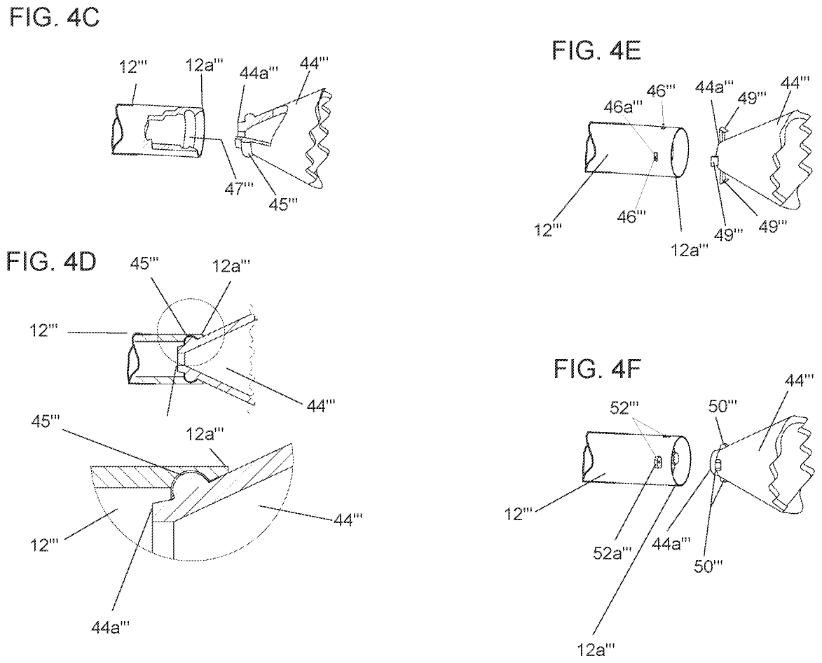

As mentioned earlier herein, one concern with the surgical environment 14 is that it is important to reduce or eliminate any contamination, such as contamination or bacteria that may be present on an outside surface 12c of the vacuum hose or tube 12. During use and in general, the sheath or drape apparatus 24 is secured to or mounted on the vacuum hose or tube 12. A user grasps and positions it in proximity to a predetermined location, such as a location near a patient or any other desired area. In general, the air handler or HVAC system 16 decontaminates contaminated air that is received from the vacuum hose or tube 12. Notice that a schematic and fragmentary view of the air handler and/or HVAC system 16′″ is shown in FIGS. 4A and 4B.

In general, the air handler and/or HVAC system 16′″ comprises a housing 16a′″ that is coupled to the second or outlet end 12b′″ of the vacuum hose or tube 12′″ as illustrated in FIGS. 4A and 4B. In general, a blower motor 16b′″ causes air to be vacuumed in the direction of arrow A in FIG. 4A and vacuumed into the vacuum hose or tube 12′″ and the housing 16a′″ of the air handler and/or HVAC system 16′″. The air travels to and through at least one or a plurality of filters 16c′″, at least one of a biocidal remover, an infrared irradiator, a chemical biocidal remover or a chemical irradiator, such as an ultraviolet tube (not shown), and after the air is filtered and decontaminated it passes through an exhaust port 16d′″ which causes the decontaminated air to be exhausted back into the surgical environment 14′″ or outside the surgical environment 14′″, such as to the outside or through the duct system 17′″, which causes the air to be exhausted to an area or outside of the surgical environment 14′″.

In the first embodiment (FIGS. 1A-1E), the vacuum hose or tube 12 is a conventional, generally cylindrical tube. Likewise, the body 18 is generally cylindrical or tubular and generally rigid, but mountable on the vacuum hose or tube 12. The sheath or drape apparatus 24 is transparent, non-gas permeable and generally cylindrical or tubular in the illustration and sized and adapted to receive and surround the vacuum hose or tube 12.

In the first embodiment (FIGS. 1A-1E), the body 18 comprises a first end 18a and a second end 18b. The first end 18a defines a first portion for coupling to the first or inlet end 12a of the vacuum hose or tube 12. The second end 18b of the body 18 provides or defines an inlet end 18b1 that defines a frusto-conically-shaped nozzle 19 that houses at least one filter 20 having a complementary shape. Notice that the end 18b defines a frusto-conically-shaped housing or support for the mating at least one filter 20, and the at least one filter 20 is frusto-conically-shaped and adapted to be received, stored or mounted in an area 22 defined by the nozzle 19. The at least one filter 20 may be disposable and secured to the nozzle 19 by suitable means that permit detachment, such as by an adhesive, press or friction fit or the like.

Thus, it should be understood that the at least one filter 20 may be a permanent filter that is permanently retained in the end 18b especially when the sheath or drape apparatus 24 is disposable after a limited-time use or it may be a single-use disposable sheath or drape apparatus 24, that is replaced or re-usable, for example, after each surgical procedure that is performed in the surgical environment 14 or any other predetermined or desired time. Alternatively, the entire sheath and drape apparatus 24 may be a limited life of use and/or disposable after a single-time use. In other words, a single-use and disposable sheath and drape apparatus 24 may be provided. Preferably, the at least one filter 20 is replaced after the sheath or drape apparatus 24 is used in the surgical environment 14 or after a predetermined time, such as after a predetermined number of uses at a predetermined time or contamination level. It should be understood that in the illustration behind described, the at least one filter 20 is not only frusto-conically shaped, but adapted to be retained in the nozzle 19 and against an internal surface 18b2 (FIG. 1C) of the end 18b by a single press or friction fit.

As air from the surgical environment 14 or around a patient enters the end 18b in the direction of arrow A (FIG. 1B), it encounters the at least one filter 20 which filters the air before it enters into the area 22 defined by the body 18 or into an area 28 (FIG. 1C) between the sheath or drape apparatus 24 and the vacuum hose or tube 12. It should also be understood that the at least one filter 20 may be integrally formed or mounted in the body 18 if the entire sheath or drape apparatus 24 is disposable. Alternatively, the body 18 may be reusable and sterilizable and have at least one of either the at least one filter 20 or the sheath or drape apparatus 24 be detachable or removable from the body 18 and disposed of and/or replaced. An important feature of the embodiments being described is that the at least one filter 20 prevents airborne contaminants from entering into the sheath or drape apparatus 24 and the other components of the surgical drape or sheath system 10 which will now be described.

As illustrated in FIGS. 1A-1E, note that the body 18 has the first end 18a that has a collar or lip 18a1 having a first end 24a of the sheath or drape apparatus 24 integrally formed therein or secured thereto. It should be understood that while the embodiment being described shows the end 24a of the sheath or drape apparatus 24 integrally formed with the collar or lip 18a1, the sheath or drape apparatus 24 could be adapted to be permanently or detachably secured thereto by a fastener or fastening means 26, such as a weld, adhesive or fastener (e.g., Velcro, weld, snaps) or the like.

After the body 18 and the first end 18a are mounted to the first or inlet end 12a of the vacuum hose or tube 12, the sheath or drape apparatus 24 may be placed or draped over the outer surface 12c of the vacuum hose or tube 12. The surface 12c of the vacuum hose or tube 12 has a diameter that is slightly smaller than an inner diameter of a surface 18c of the body 18 in the embodiment of FIGS. 1A-1E so that the end 12a can be inserted into area 22 defined by the body 18 so that the first or inlet end 12a and surface 12c engage and become secured to the surface 18c of the body 18 with a press or friction fit, as illustrated in FIG. 1C.

It should be understood that after the end 12a of the vacuum hose or tube 12 is mounted in the body 18, the sheath or drape apparatus 24 is placed or draped over the end 12a of the vacuum hose or tube 12 as illustrated in FIG. 1C. For ease of illustration, FIG. 1C shows the sheath or drape apparatus 24 draped over the end 12a of the vacuum hose or tube 12. A retention strap, ring, tie, collar or other fastener or fastening means 26 may also be applied to a second end 24b of the sheath or drape apparatus 24 or to the surface 12c of the vacuum hose or tube 12 as shown. The sheath or drape apparatus 24 cooperates with the surface 12c to define the closed area 28 between the outer surface 12c or in the area 22 of the vacuum hose or tube 12 and an inner surface 24c of the sheath or drape apparatus 24. It should be understood that at no time does ambient air or contaminated air that is received in the area 22 of the body 18 flow into the area 28. Also, no air or outer surface 12c covered by the sheath or drape apparatus 24 is exposed to the surgical environment 14 or the patient. This feature prevents any contamination on the surface 12c from being transmitted to a person or into the surgical environment 14. Thus, the sheath or drape apparatus 24 functions to prevent any contamination from the surface 12c or from the area 28 from leaving or exiting the area 28. In the illustration being described, the fastener or fastening means 26 may be a Velcro strap, tie, elastic band or an integrally formed tie or other means for securing the end 24b of the sheath or drape apparatus 24 against the surface 12c of the vacuum hose or tube 12.

As illustrated in FIGS. 1A-6C, note that both the body 18 and the sheath or drape apparatus 24 are flexible, impermeable and may be transparent as shown. In the first embodiment (FIGS. 1A-1E) being described, the sheath or drape apparatus 24 is generally tubular, flexible and collapsible, similar to a conventional plastic garbage bag or tubular sock so that it may be adapted and spread over the surface 12c of the vacuum hose or tube 12 as illustrated. The fastener or fastening means 26 may be integrally formed in the sheath or drape apparatus 24 or may be a separate component that is adapted to secure and seal the end 24b of the sheath or drape apparatus 24 against the surface 12c of the vacuum hose or tube 12.

During use, the end 12a of the vacuum hose or tube 12 is inserted into the end 18a of the body 18 and press-fit into the end 18a so that the surface 12c engages and is secured to the surface 18c with a press-fit or friction fit. Preferably, the press-fit provides a friction fit and an airtight seal that prevents contaminated air from leaking out of the area 28 between the surfaces 12c and 18c. If contaminated air should leak between the surfaces 12c and 18c and into the area 28, the fastener or fastening means 26 prevent the contaminated air from leaking out of the area 28, such as between the engagement of the inner surface 24c and the surface 12c of the vacuum hose or tube 12.

After the vacuum hose or tube 12 has been received in the generally cylindrical and tubular sheath or drape apparatus 24 and the end 12a inserted into the end 18a of the body 18, the sheath or drape apparatus 24 is extended in the direction of arrow B (FIG. 1C) so that it drapes over the surface 12c of the vacuum hose or tube 12 for a predetermined length (double arrow PL in FIG. 1C). It should be understood that the sheath or drape apparatus 24 seals and prevents any contamination on the surface 12c of the vacuum hose or tube 12 from entering the surgical environment 14 or from contaminating a person (such as a nurse, doctor or patient) in the surgical environment 14. The sheath or drape apparatus 24 comprises an outer surface 24d that is sterile and not contaminated so that a user, such as the nurse or doctor, may grasp the vacuum hose or tube 12 so that the end 18b may be directed toward, for example, an area around the patient to be aspirated or a surgical site. Alternatively, the user may grasp the sheath or drape apparatus 24 and an outer surface 18d of the body 18 for purposes of directing the end 18b toward the surgical site.

During a surgical procedure, the sheath or drape apparatus 24 is mounted onto the vacuum hose or tube 12 in the manner described herein prior to the surgical procedure. While the embodiment has been shown as having the first or inlet end 12a inserted into the end 18a, it should be understood that these components could be adapted so that the end 18a is inserted into the first or inlet end 12a of the vacuum hose or tube 12. Prior to or at this time, the at least one filter 20 may be checked and if contaminated, replaced prior to the surgical procedure in the surgical environment 14. Once the at least one filter 20 is checked, it may be replaced with a new or recycled at least one filter 20, which is press-fit into place into the nozzle end 18b of the body 18. The at least one filter 20 is mounted into the nozzle end 18b of the body 18, and the sheath or drape apparatus 24 is moved or draped in the direction of arrow B (FIG. 1C) over the vacuum hose or tube 12. The fastener or fastening means 26 is tied or mounted on the sheath or drape apparatus 24 and the vacuum hose or tube 12 to seal the sheath or drape apparatus 24 against the vacuum hose or tube 12 is ready for use during a surgical procedure in the surgical environment 14.

FIGS. 1D and 1E are sectional views showing further details of the components described herein.

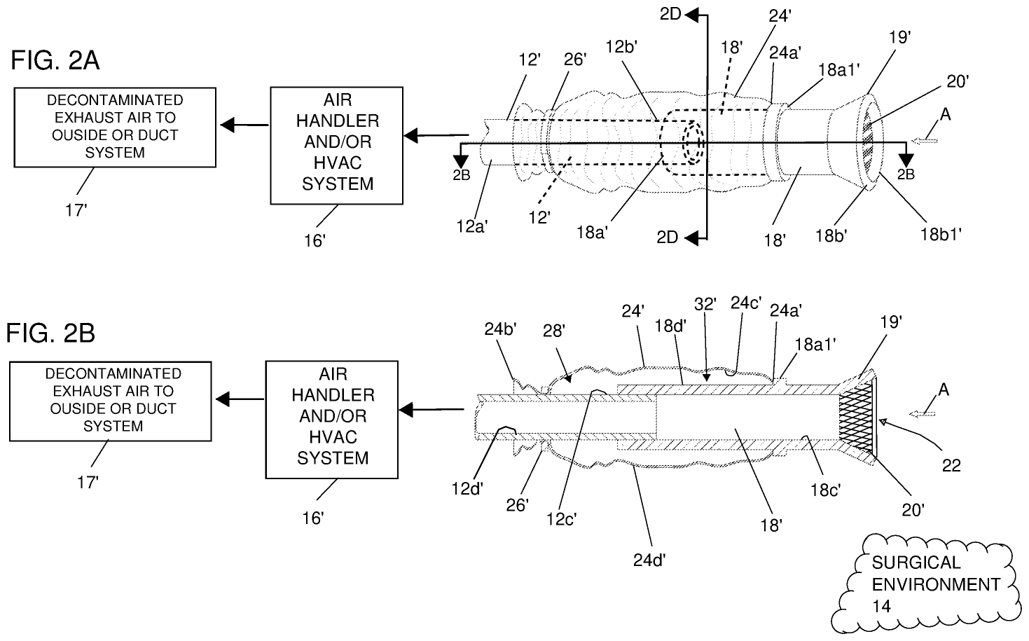

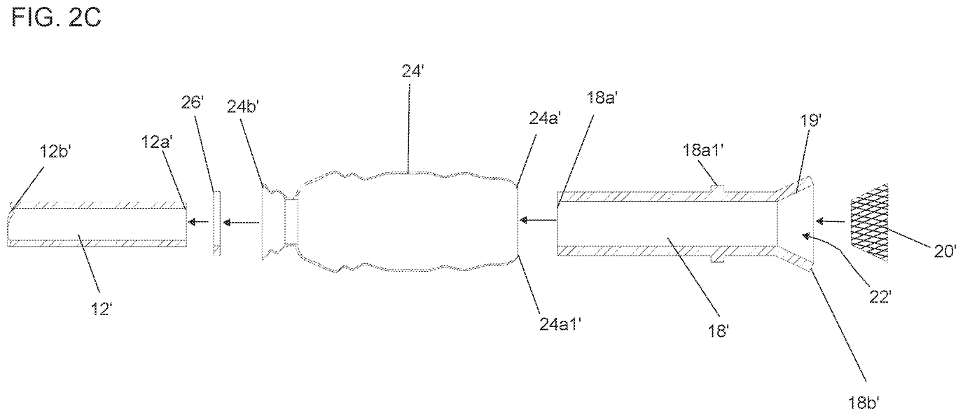

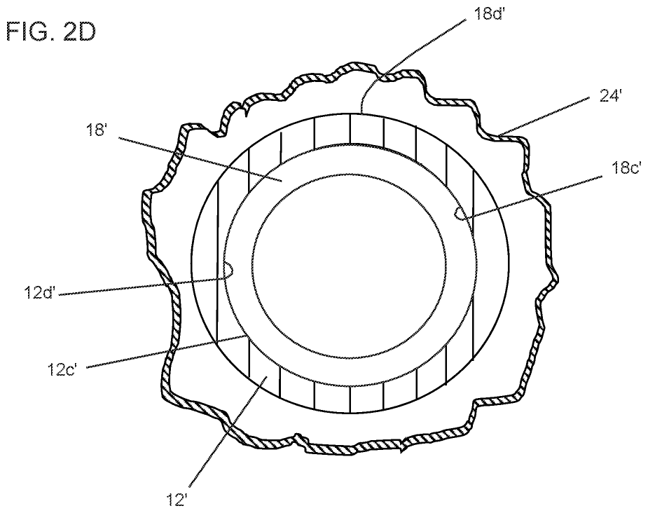

FIGS. 2A-2D illustrate a second embodiment of the surgical drape or sheath system that function similarly to the surgical drape or sheath system 10 shown in FIGS. 1A-1E. As mentioned earlier, during use, the user grasps the sterile sheath or drape apparatus 24 and the vacuum hose or tube 12 or body 18 and positions it in proximity to a predetermined location, such as a location near the patient or any other desired area. As mentioned earlier, the same parts are identified with the same part numbers, except that one prime mark has been added to these like part numbers for the second embodiment. In short, the second embodiment illustrates and describes a single-use disposable flexible tubular sheath or drape apparatus 24 that is placed over the vacuum hose or tube 12 in order to facilitate preventing or reducing the risk of any contaminants or bacteria on the vacuum hose or tube 12 from being transferred to a person or into the surgical environment 14. The second embodiment will now be described.

Notice in FIGS. 2A-2D that the system 10′ is similar except that the collar or lip 18a1′ is integrally formed and/or located closer to the nozzle 19′ as illustrated. Notice that the sheath or drape apparatus 24′ extends not only over the area 28′ defined by the area between the inner surface 24c′ of the sheath or drape apparatus 24′ and the surface 12c′ of the vacuum hose or tube 12′. Notice also that the sheath or drape apparatus 24′ extends over an area 32′ between the inner surface 24c′ of the sheath or drape apparatus 24′ and the outer surface 18d′ of the body 18′, thereby covering not only at least a portion of the surface 12c′ of the vacuum hose or tube 12′, but also at least a portion of the surface 18d′ of the body 18′.

Advantageously, this embodiment facilitates draping the sheath or drape apparatus 24′ over the surfaces 12c′ and 18d′ in order to prevent contamination that may reside on these surfaces from being transferred to a person or into the surgical environment 14′. In the illustration being described, the sheath or drape apparatus 24′ is integrally formed with the collar or lip 18a1′. Similar to the prior embodiment of FIGS. 1A-1E, the first end 24a′ is integrally formed in the collar or lip 18a1′ or is secured thereto by other means, such as a weld, adhesive, fastener (e.g., Velcro, weld, snaps) or the like. Notice how the sheath or drape apparatus 24′ in FIG. 2B extends over at least a portion of the surfaces 18d′ and 12c′ as described and illustrated. After the end 24b′ is properly positioned relative to the vacuum hose or tube 12′, the fastener or fastening means 26′, the Velcro strap or other integrally formed tie or other means may be used to secure the end 24b′ of the sheath or drape apparatus 24′ against the surface 12c′ of the vacuum hose or tube 12′, thereby sealing the areas 28′ and 32′. This facilitates preventing contamination in those areas and on the surfaces 12c′ and 18d′ from being transmitted to a person who handles either the vacuum hose or tube 12′ or the body 18′. It also facilitates preventing any contamination on the surfaces 12c′ and 18d′ from being transmitted into the surgical environment 14′. FIG. 2D is a sectional view showing further details of the components described herein.

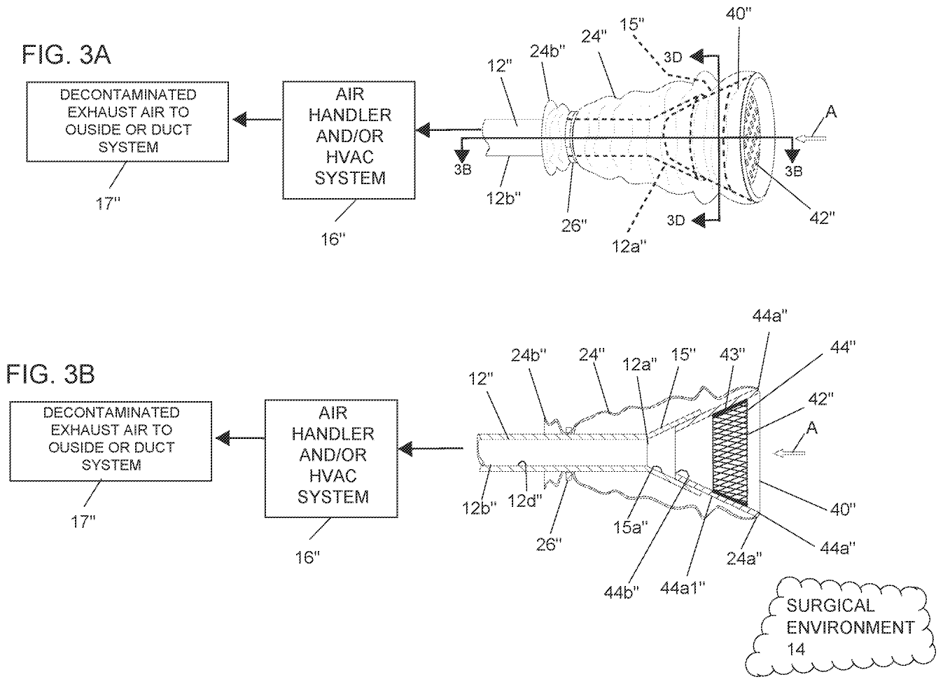

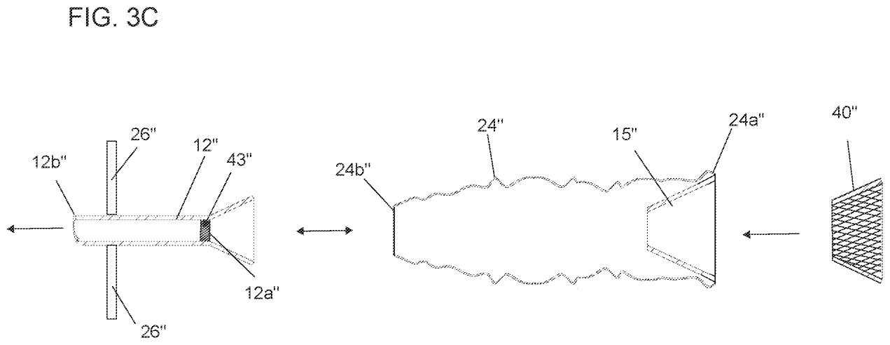

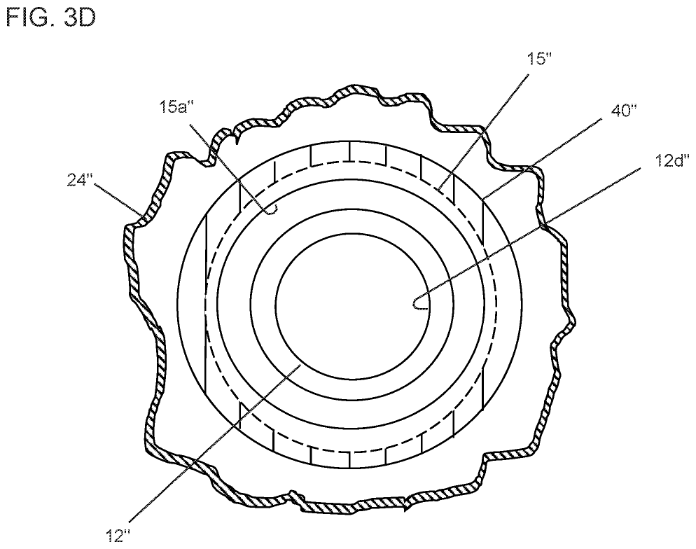

Referring now to a third embodiment illustrated in FIGS. 3A-3D, another body 18″ and sheath or drape apparatus 24″ are illustrated. In this embodiment, notice that the surface 12a″ of the vacuum hose or tube 12″ comprises a frusto-conically shaped nozzle 15″ that is adapted to receive a filter 40″. In this embodiment, the filter 40″ is also frusto-conically shaped as illustrated in FIGS. 3A-3D and comprises a filter element or core 42″ that is mounted in a body 44″ as shown. In this regard, it should be appreciated that the filter 40″ may be a disposable filter or may be the permanent body 44″ that is adapted to receive the removable filter element or core 42″. In one illustration, the body 44″ is made from a non-gas permeable paper or cellular material and the filter element or core 42″ is secured thereto by conventional means, such as a weld, adhesive, press-fit or the like. Notice that the sheath or drape apparatus 24″ is integrally formed or coupled to an end 44a″ of the body 44″ as shown. As with prior embodiments, the sheath or drape apparatus 24″ is integrally formed at its end 24a″ to the end 44a″ or is fastened thereto by conventional means, such as a weld, adhesive or other fastener or fastening means. Notice that as with prior embodiments, the sheath or drape apparatus 24″ is draped over both the filter body 44″, the nozzle 15″ and at least a portion of the surface 12a″ of the vacuum hose or tube 12″. As with prior embodiments, the fastener or fastening means 26″ secures the sheath or drape apparatus 24″ to the vacuum hose or tube 12″ as shown.

It should be understood that the body 18″ and its integrally-formed sheath or drape apparatus 24″ are disposable and intended for one-time use in this embodiment. Prior to a surgical procedure in the surgical environment 14″, the filter body 44″ is inserted and press-fit into the end of the nozzle 15″ so that a surface 44a1″ and an interior surface 15a″ of the nozzle 15″ engage and the surface 15a″ and becomes secured in the nozzle 15″ by press-fit or friction fit which retains the filter body 44″ in the nozzle 15″.

During use, the user grasps the sheath or drape apparatus 24″ and positions it in proximity to a predetermined location, such as a location near the patient or any other desired area. The sheath or drape apparatus 24″ facilitates preventing or reducing any contamination being transferred, for example, from the surfaces 44a1″ and 12a″ to the patient or the surgical environment 14″. FIG. 3D is a sectional view showing further details of the components described herein.

Referring now to FIGS. 4A-4B, the embodiment of FIGS. 4A-4B is substantially similar to the embodiment of FIGS. 3A-3B, except that the end 12a′″ is not frusto-conically shaped as shown. The filter body 44′″ is press-fit into the end 12a′″ of the vacuum hose or tube 12′″ as shown. In this regard, the end 44a′″ of the filter body 44′″ may have ridges or serrations there around to facilitate retaining the filter body 44′″ in the end 12a′″ of the vacuum hose or tube 12′″. Thus, an interior edge or corner edge 12a1′″ engages the surface 44a1′″. FIG. 4C illustrates an embodiment where the body 44′″ has at least one or a plurality of raised ridges 45 that cooperate with the corner edge 12a1′″ to facilitate retaining an end 44c′″ of the body 44′″ in the end 12a′″ of the vacuum hose or tube 12′″ as illustrated.

If the embodiment is provided with at least one or a plurality of ridges 45′″, then the corner edge 12a1″″ of the vacuum hose or tube 12′″ is guided over or snapped over the at least one or a plurality of raised ridges 45′″ and provides a retainer or retaining means for retaining and securing the body 44′″ into the end 12a′″ of the vacuum hose or tube 12′″. Of course, other devices and means may be used for detachably retaining the filter 40″″ in the vacuum hose or tube 12′″, such as a non-permanent adhesive, snaps and the like.

For both of the embodiments in FIGS. 3A-4F, the filter body 44″″ is mounted onto the nozzle 19′″ in the manner described herein. As previously mentioned, the adhesive, weld or other type of fastener or fastening means 26′″ secures the body 44′″ onto the vacuum hose or tube 12′″. FIGS. 4C-4F Illustrate other suitable means for attaching the body 44′″ of the filter 40′″ to the vacuum hose or tube 12′″ in order to detachably secure the body 44′″ to the vacuum hose or tube 12′″ as shown. Notice in FIG. 3B, for example, that the body 44″ has an adhesive 43″ thereon so that when the body 44″ is inserted into the nozzle 15″ of the vacuum hose or tube 12″, it is detachably adhered thereto. Various other suitable means for fastening the body 44″ onto the vacuum hose or tube 12″ could also be used and a few illustrative examples will now be described relative to FIGS. 4D-4F.

Notice in FIG. 4C that the at least one or a plurality of raised ridges 45′″ is integrally formed at or near the end 44a′″. The at least one or a plurality of raised ridges 45′″ are adapted and complementary in shape to mating apertures or grooves 47′″ which resides in the end 12a′″ of the vacuum hose or tube 12′″ so that when the body 44′″ and the end 44a′″ are inserted into the end 12a′″ of the vacuum hose or tube 12′″, the at least one or a plurality of raised ridges 45′″ is received in the groove 47′″ and thereby secures the body 44′″ to the vacuum hose or tube 12′″.

FIG. 4E illustrates another fastener or fastening means for securing the end 44a′″ of the body 44′″ to the end 12a′″ of the vacuum hose or tube 12′″. In this embodiment, the end 12a′″ comprises a plurality of walls 46a′″ that define a plurality of apertures 46′″ that are adapted and shaped to receive a plurality of latches 49 that are integrally formed with the end 44a′″ of the body 44′″. When the end 44a′″ is inserted into the end 12a′″ of the vacuum hose or tube 12′″, the male latches 49′″ are matingly and adaptively received in the female apertures 46′″, thereby locking the body 44′″ to the vacuum hose or tube 12′″.

In the embodiment of FIGS. 4C and 4D, a boss or raised ridge 45′″ is used in cooperation with a female groove 47′″ on the vacuum hose or tube 12′″. Referring now to FIG. 4F, notice that a plurality of raised ribs or bosses 50″″ are integrally formed in the end 44a′″ of the body 44′″. The plurality of raised ribs or bosses 50′″ are defined male projections that are shaped and adapted to complement the shape of mating female depressions, areas or apertures 52′″ which are each defined by an aperture wall, 52a′″, respectively and that are complementary in shape to the plurality of raised ribs or bosses 50′″. When the end 44a′″ is inserted into the end 12a′″ of the vacuum hose or tube 12′″, the plurality of raised ribs or bosses 50″″ are received in the mating female, depressions, areas or apertures 52′″ to secure the end 44a′″ of the body 44′″ to the end 12a′″ of the vacuum hose or tube 12′″.

Advantageously, the surgical drape or sheath system 10 and the various embodiments illustrate different fasteners and means for fastening or securing the at least one filter 20 or filter 40 to the body 18 or vacuum hose or tube 12 in the manner illustrated. These fasteners or fastening means 26 may be used with any embodiment. As mentioned earlier herein, the at least one filter 20 is detachably secured to the nozzle 19 of the body 18 and the body 18 is secured to the vacuum hose or tube 12, as illustrated in the embodiments of FIGS. 1A-2D, or the body 18″ is mounted onto the vacuum hose or tube 12′″, as illustrated in the embodiments of FIGS. 3A-4F. Then, the sheath or drape apparatus 24′″ is situated or draped over at least a portion of the body 18′″ and body 44′″ as well as at least a portion of the vacuum hose or tube 12′″, and the fastener or fastening means 26″″ is used to secure the end 24b′″ of the sheath or drape apparatus 24′″ to the vacuum hose or tube 12′″ as illustrated. The user may then grasp the sheath or drape apparatus 24′″ and position the nozzle 19′″ or the end 18b′″ in the predetermined location, which may be adjacent to or near the patient in the surgical environment 14′″.

Although not shown, it should be appreciated that the embodiments of FIGS. 1A-2D may utilize a threaded connection in which the end 12a of the vacuum hose or tube 12 comprises a male thread and the end 18b of the body 18 comprises a mating female thread so that the body 18 may be threadably received on the end 12a of the vacuum hose or tube 12. Again, in a preferred embodiment, the body 18′″ is detachably secured to the vacuum hose or tube 12′″ and, likewise, the body 44′″ of the filter 40′″ is detachably press-fit or secured to the nozzle 15′″ of the vacuum hose or tube 12′″ for the embodiments illustrated in FIGS. 3A-4F.

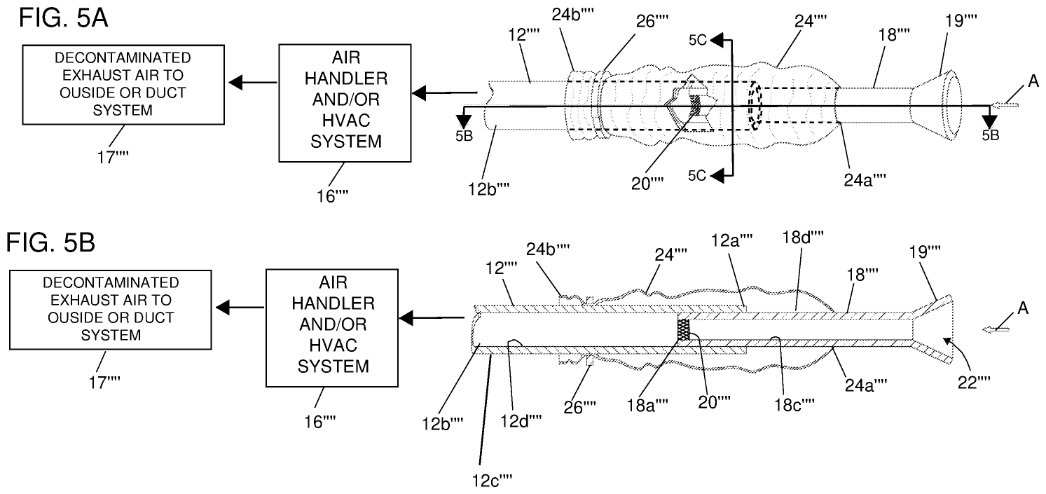

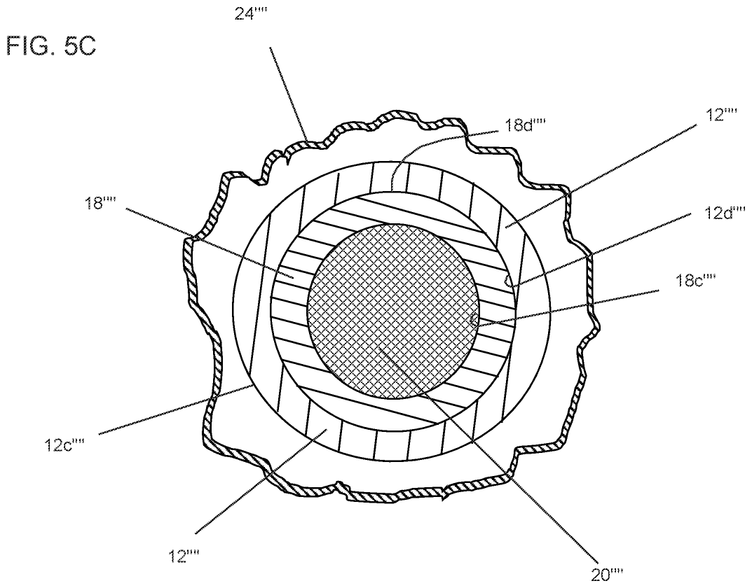

FIGS. 5A-5C illustrate another embodiment of the surgical drape or sheath system 10″″. In this embodiment, the end 24a″″ of the sheath or drape apparatus 24″″ is integrally formed or secured to the surface 18d″″ of the body 18″″ as illustrated. Note that the at least one filter 20″″ in this embodiment is not located in the nozzle 19″″, but rather is situated in the area 22″″ toward the downstream outlet end 18a″″ of the body 18″″. In the illustration being described, the at least one filter 20″″ is detachably received in and engages the surface 18c″″ of the body 18″″, thereby securing the detachable filter 20″″ thereto. As with prior embodiments, the detachable filter 20″″ may be press-fit into the end 18a″″ or may be detachably secured to by another fastener or fastening means such as a weld, adhesive or other type of fastener or fastening means 26″″ as described herein. In this embodiment, the end 18a″″ is press-fit in the vacuum hose or tube 12″″ and is retained therein by the friction-fit between the surface 18d″″ of the body 18″″ and the surface 12c″″ of the vacuum hose or tube 12″″. As with prior embodiments, this may be a press-fit or friction fit connection, a threaded connection, a connection of the type illustrated and shown and described relative to FIGS. 4D-4F or any other suitable fastener or fastening means adapted to secure the body 18″″ to the vacuum hose or tube 12″″.

Moreover, it should be appreciated that the body 18″″ and the vacuum hose or tube 12″″ may be threadably coupled together. If a threaded connection were used, for example, a male thread (not shown) is situated on the outer surface 18d″″ of the end 18a″″ and a mating female thread (not shown) is situated on the surface 12c″″ of the vacuum hose or tube 12″″, thereby permitting the body 18″″ to be threadably mounted and secured to the vacuum hose or tube 12″″. After the body 18″″ is mounted on the vacuum hose or tube 12″″, the sheath or drape apparatus 24″″ is draped over at least a portion of the surfaces 18d″″ of the body 18″″ and surface 12c″″ of the vacuum hose or tube 12″″ as illustrated.

Advantageously, with this embodiment the risk of a user touching or coming into contact with the at least one filter 20″″ is eliminated or substantially reduced. In this regard, while it is preferred that the at least one filter 20″″ be disposable and changed out periodically, it is not uncommon that the at least one filter 20″″ may not be contaminated to the point where either the at least one filter 20″″ or the entire body 18″″ needs to be replaced. In such circumstances, the at least one filter 20″″ may still retain or still have some contamination or bacteria thereon and so it is important to locate the at least one filter 20″″ away from the nozzle 19″″ so that it does not come into contact with any person, place, the surgical environment 14″″ or the patient. By situating the at least one filter 20″″ downstream of the nozzle 19″″, as illustrated in FIGS. 5A-5C, the risk of contamination from the at least one filter 20″″ is substantially reduced or eliminated.

As with the prior embodiments, once the at least one filter 20″″ is situated in the body 18″″ and the body 18″″ is mounted to the vacuum hose or tube 12″″, the nozzle 19″″ can be situated by the user at the predetermined location which may be adjacent to or near the patient in the surgical environment 14″″. FIG. 5C is a sectional view showing further details of the components described herein.

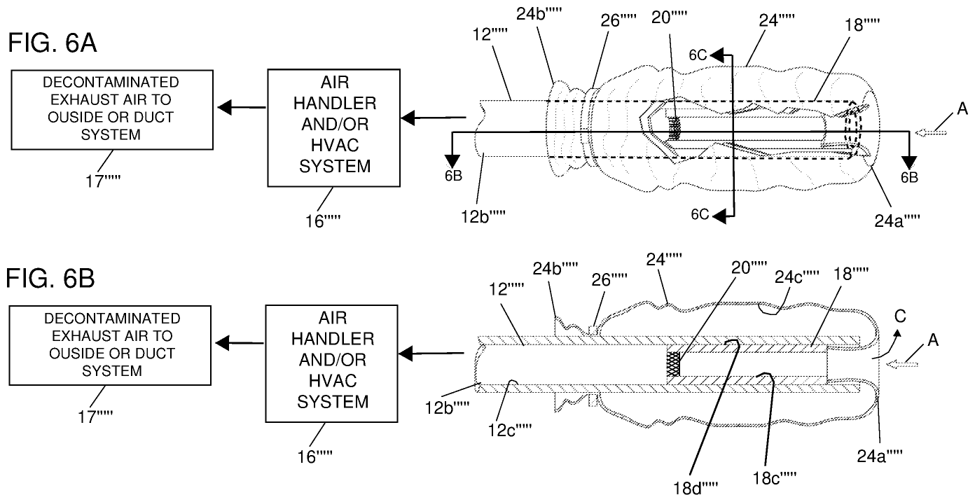

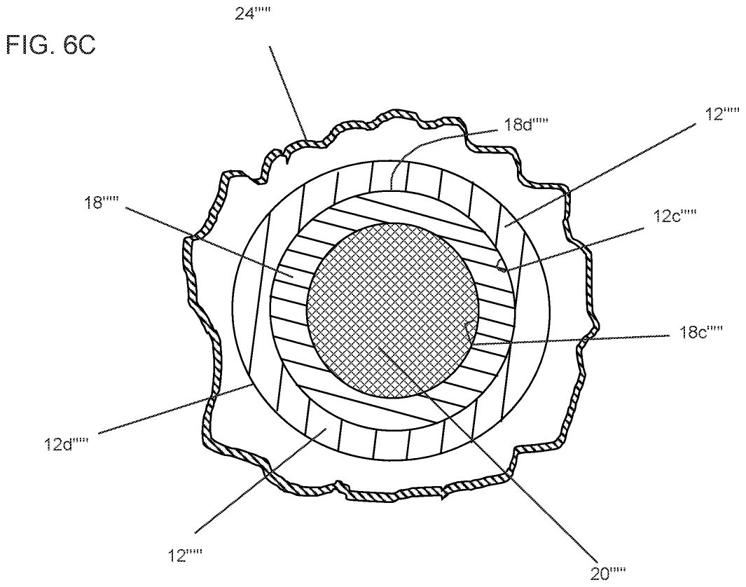

Referring now to FIGS. 6A-6C, still another embodiment is shown. In this embodiment, notice that the body 18″″″ is completely received and enveloped inside the vacuum hose or tube 12′″″. In this regard, the outer surface 18d″″″ of the body 18″″″ has a slightly smaller diameter than the inner diameter of the vacuum tube 12′″″, so that the body 18″″″ itself can be inserted completely in the end 12a′″″ of the vacuum hose or tube 12″″′. Notice that the location of the at least one filter 20″″″ in this embodiment is similar to the embodiment shown and described relative to FIGS. 5A-5C in that the at least one filter 20″″″ is located in the end 18b″″″ of the body 18″″″ as illustrated. Notice that the end 24a′″″ of the sheath or drape apparatus 24″″′ is secured to or integrally formed in the end 18b″″″ of the body 18″″″. As with the embodiment shown and described relative to FIGS. 5A-5C, notice that the at least one filter 20″″″ is press-fit into the end 18a″″″ of the body 18″″″ as shown. The advantages mentioned earlier herein relative to FIGS. 5A-5C regarding the location of the at least one filter 20″″″ also apply to this embodiment.

Notice that after the body 18″″″ is press-fit or detachably inserted into the end 12a″″″ of the vacuum hose or tube 12″″″, the drape or sheath apparatus 24″″″ is draped or positioned over a large portion of the surface 12c″″″ as illustrated. Once the sheath or drape apparatus 24″″″ is positioned over at least a portion of the surface 12c″″″ of the vacuum hose or tube 12″″″, then the fastener or fastening means 26″″″ may be used to secure the end 24b″″″ of the sheath or drape apparatus 24″″″ directly to the surface 12c″″″ of the vacuum hose or tube 12″″″.

After the sheath or drape apparatus 24″″″ is positioned and fastened, the user may grasp the sheath or drape apparatus 24″″″ and position the end 12a″″″ of the vacuum hose or tube 12″″″ in proximity to the predetermined location which, again, is a location preferably by or near the patient in the surgical environment 14″″″.

Advantageously, with this embodiment no portion of the body 18″″″ extends outside or beyond an end 12a″″″ of the vacuum hose or tube 12″″″, thereby reducing or eliminating the chance that this component and, particularly, the outer surface 18d′″″ of the body 18″″′ are all housed inside the vacuum hose or tube 12′″″, while the non-gas permeable sheath or drape apparatus 24″″″ is draped outwardly (in the direction of arrow C in FIG. 6B) and until the end 24b″″″ of the sheath or drape apparatus 24″″″ is situated where desired over the vacuum hose or tube 12″″″ and the fastener or fastening means 26″″″ used to secure the end 24b″″″ to the vacuum hose or tube 12″″″ as shown. The sheath or drape apparatus 24″″″ form a general U-shape in cross-section as viewed in FIGS. 6A-6C. FIG. 6C is a sectional view showing further details of the components described herein.

This embodiment, as well as the previous embodiments described and shown, provide for allowing a non-sterile vacuum hose or tube 12″″″ to be safely used in a sterile surgical procedure in the surgical environment 14″″″. Due to the flexible nature of the vacuum hose or tube 12′″″ used in the past, the surgical drape or sheath system 10″″′ provides for a flexible, impermeable surgical drape component comprising the body 18″″″ and the sheath or drape apparatus 24″″″. The sheath or drape apparatus 24″″″ functions to prevent the spread of contamination and bacteria between the vacuum hose or tube 12″″″ and either a person, patient or a surgical site in the surgical environment 14″″″. It should also be understood, however, that the surgical drape or sheath system 10″″″ comprises the gas-permeable component in the form of the filters 20″″″ and 40″″″ and filter bodies 18″″″ and 44″″″ permit gas to pass therethrough. This allows for vacuumed air to be suctioned into and through the vacuum hose or tube 12″″″ so that it can be received by the air handler and/or HVAC system 16″″″ and decontaminated and then expelled into the surgical environment 14″″″ or to other areas, such as outside the surgical environment 14″″″ or into the duct system 17″″″ of a building (not shown).

### Additional Considerations

1. In the illustration being described, the sheath or drape apparatus 24 may comprise a polymer, paper, textile or other flexible and substantially impervious material that is non-gas permeable. In the illustrations being described, the material is preferably capable of being sterilized for use in a surgical procedure, and the sheath or drape apparatus 24 are also transparent so that the components inside the sheath or drape apparatus 24 and the positioning of the body 18 relative to the vacuum hose or tube 12 may be observed.

2. As illustrated in the various embodiments, the sheath or drape apparatus 24 is not only transparent, but generally tubular and incorporates the body 18 which may be viewed as an elongated sterile extension that creates a sterile transition zone between the surgical site and the vacuum hose or tube 12. Some embodiments show a single-use disposable, flexible tubular sheath or drape apparatus 24 placed over the vacuum hose or tube 12 and incorporating the concave or conical air filter 20 or filtration means in proximity to the nozzle 19 of the vacuum hose or tube 12, while other embodiments illustrate the sheath or drape apparatus 24 positioned in a U-shaped manner (when viewed in cross-section) as illustrated in FIGS. 6A-6C, such that the body 18 extends substantially into the interior of the vacuum hose or tube 12 and terminates in a gas-permeable region and thereby maintaining a sterile transition zone between the vacuum hose or tube 12 and the surgical site in the surgical environment 14. In this regard, note that when the embodiment of FIGS. 6A-6C is used in the surgical environment 14, both the body 18 and the surface 12c of the vacuum hose or tube 12 cannot directly contact a person, place, thing or a patient in the surgical environment 14 because it is covered by the sheath or drape apparatus 24 as illustrated. If the end 12a of the vacuum hose or tube 12 is positioned at the predetermined location, which is preferably near the patient in the surgical environment 14, the body 18 and at least a portion of the surface 12c is covered by the sheath or drape apparatus 24 so that contamination cannot be transferred from either component to the patient, nurse, doctor or the surgical environment 14. When the vacuum hose or tube 12 of the embodiment of FIGS. 6A-6C is used in the surgical environment 14, the sheath or drape apparatus 24 creates a barrier between the patient and/or the surgical environment 14 and the contaminated surface 12c of the vacuum hose or tube 12.

3. In the illustration being described, the sheath or drape apparatus 24 is made from an impermeable flexible polymer, such as polyethylene, low-density polyethylene (LDPE) polypropylene. poly(tetrafluoroethylene) or silicones. Of course other types of materials may be used provided they provide the impermeability desired. It is also preferable that the material be transparent, although that is not required. With a transparent sheath or drape apparatus 24, the assembly of the body 18 to the vacuum hose or tube 12 and the visibility of the other components described herein is enhanced. Again, however, it is not mandatory.

4. In the illustration being described, the body 18 is manufactured from substantially rigid and non-gas permeable polymer, such as high-density polyethylene (HDPE) polypropylene (PP), polystyrene (PS), Nylon, thermoplastic polyurethanes (TPU), or acrylics. Again, while these are preferred materials for manufacturing the body 18, other materials could be used, such as cellular materials, paper, textiles or the like.

5. The system 10 and method herein describe and provide a process and method for preventing contamination of a vacuum hose or tube 12 used in a surgical operating room, the vacuum hose or tube 12 comprising the distal end 12a, the method comprising the steps of placing a substantially tubular sheath over the vacuum hose or tube 12, the substantially tubular sheath or drape apparatus 24 comprising a filtration end having a filter 20, coupling the filtration end 18a of the body 18 to the distal end 12a so that air passing into the vacuum hose or tube 12 gets filtered by the filter 20.

6. It should be understood that the body 18 and the sheath or drape apparatus 24 may be integrally provided together or the sheath or drape apparatus 24 may be detachable from the body 18 and disposable, so that the body 18 may be reusable, while the sheath or drape apparatus 24 is disposable. Alternatively, the sheath or drape apparatus 24 may be sterilized for use in a surgical environment.

7. In the embodiments shown, a disposable filter 20 may be mounted in an end of at least one of the body 18 or the vacuum hose or tube 12 and the filter 20 may comprise a shape, such as a frusto-conical shape that mates with an end 18a of the body 18 which may comprise a nozzle 19 having a complementary frusto-conical shape.

8. As mentioned previously herein, the surgical drape or sheath system, apparatus and method described herein accomplishes various objects including:
providing a surgical drape or sheath system for use in a medical environment;
providing a surgical drape or sheath system and method that facilitates preventing or reducing a transfer of contaminants to a component in a surgical environment to a person, such as a nurse, doctor, patient or the like or to the environment in which the patient is located, such as patient room or surgical operating room;
providing a sheath and drape system and method that comprises a body having a flexible and non-permeable drape or sheath that can be mounted on a vacuum tube that is used in a surgical environment;
providing a surgical drape or sheath having a body that is mounted on a vacuum tube and that has a flexible drape and sheath component that is generally tubular and that can be draped over the vacuum tube in order to reduce or eliminate the chance of any bacteria or contamination on the vacuum tube from being transferred to a person or into the local environment;
providing a sheath and drape system and method that comprises a body that is mounted on an end of the vacuum tube;
providing a sheath and drape system that comprises a body and a flexible sheath and drape wherein the body is mounted inside the vacuum tube and completely concealed by the vacuum tube while the drape is draped over the outer surface of the vacuum tube;
providing a sheath and drape system having a body that is mounted inside the vacuum tube, while other embodiments provide means and apparatus for mounting the body on the outside of the vacuum tube;
providing a sheath and drape system having a filter located in a nozzle end of the body, with the nozzle end and the filter having a complementary shape and in at least some embodiments being frusto-conical in cross-section;
providing a sheath and drape system wherein the body comprises a frusto-conical shape and is matingly received in an end of the vacuum tube which also comprises a mating frusto-conical shape;
providing means and apparatus for securing the body of the surgical drape or sheath system to a vacuum tube; and
providing a surgical drape or sheath system that is sterilizable and re-usable or is disposable, for example, after a one-time use.

Advantageously, another embodiment of this invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. A surgical sheath for use with a vacuum device having a vacuum tube, said surgical sheath comprising:
a body having an open end and a second end, said body being adapted and sized to be coupled to or in fluid communication with an end of said vacuum tube;
a sheath coupled to said second end of said body and adapted to receive at least a portion of said vacuum tube; and
a filter for mounting to said second end of said body and filtering air that flows therethrough, said body not permitting airflow peripheral to said filter and said sheath and said body cooperating to substantially prevent air movement between said sheath and said vacuum tube after said body is detachably mounted thereto.

2. The surgical sheath as recited in claim 1, wherein said body is impermeable to air and said filter is mounted on said second end to filter air passing into said vacuum tube.

3. The surgical sheath as recited in claim 2, wherein openings of said filter have a diameter of less than or equal to 1 mm in diameter.

4. The surgical sheath as recited in claim 1, wherein said filter comprises a substantially convex conical shape.

5. The surgical sheath as recited in claim 1, wherein said filter comprises a generally conical shape, said open end being sized, dimensioned and adapted to have a generally complementary shape as said filter and adapted to receive and support said filter so that it filters the air passing into said vacuum tube.

6. The surgical sheath as recited in claim 1, wherein said body and said vacuum tube comprises a connection for providing a substantially air-tight seal between said open end and said vacuum tube.

7. The surgical sheath as recited in claim 6, wherein said connection is reversible and comprising complementary shaping, tapering, fastening, compressing, plugging, sliding, pinning, keying, threading, rotating, magnetically attracting, hook and loop connecting, or comparable means.

8. The surgical sheath as recited in claim 6, wherein said filter comprises open-cell foam, sieve, textile, paper, fiber or similar air filtration means;
said sealing comprising a substantially air-tight border comprising heat seal, adhesive, fastener or similar joining means.

9. The surgical sheath as recited in claim 1, wherein said surgical sheath is manufactured from impermeable flexible polymer, such as polyethylene, low-density polyethylene (LDPE) polypropylene. poly(tetrafluoroethylene) or silicones.

10. A method for preventing contamination of a vacuum hose used in a surgical operating room, said vacuum hose comprising a distal end, said method comprising the steps of:
placing a substantially tubular sheath over said vacuum hose, said substantially tubular sheath comprising a filtration end having a filter;
coupling said filtration end to said distal end so that air passing into said vacuum hose gets filtered by said filter.

11. The method as recited in claim 10, wherein said substantially tubular sheath comprises an air-impermeable body adapted to be mounted on said distal end of said vacuum hose and a non-permeable sheath portion for facilitating preventing contamination of an external surface of said vacuum hose.

12. A surgical sheath for use with a medical vacuum device having a vacuum end, said surgical sheath comprising:
a substantially flexible body that becomes situated around at least a portion of said medical vacuum device;
a substantially rigid extension having a coupling end for detachably mounting to said vacuum end such that said substantially flexible body becomes situated around at least a portion of said medical vacuum device;
a filter situated at said coupling end of said substantially rigid extension for filtering air before it enters said medical vacuum device.

13. The surgical sheath as recited in claim 12 wherein said filter is generally conically shaped and is situated in said coupling end of said substantially flexible body.

14. The surgical sheath as recited in claim 12 wherein said substantially rigid extension is impermeable to air and said filter is mounted on said coupling end to filter air passing into said medical vacuum device.

15. The surgical sheath as recited in claim 14 wherein openings of said filter have a diameter of less than or equal to 1 mm in diameter.

16. The surgical sheath as recited in claim 12 wherein said filter comprises a substantially convex conical shape.

17. The surgical sheath as recited in claim 12 wherein said filter comprises said coupling end being sized, dimensioned and adapted to have a generally complementary shape as said filter and adapted to receive and support said filter so that said filter filters the air passing into said vacuum device.

18. The surgical sheath as recited in claim 12 wherein said medical vacuum device comprises a connection for providing a substantially air-tight seal between substantially rigid extension and said vacuum device.

19. The surgical sheath as recited in claim 18 wherein said connection is reversible and comprising complementary shaping, tapering, fastening, compressing, plugging, sliding, pinning, keying, threading, rotating, magnetically attracting, hook and loop connecting, or comparable means.

20. The surgical sheath as recited in claim 12 wherein said coupling comprises a filter which is sealed to said coupling;
said filter comprises open-cell foam, sieve, textile, paper, fiber or similar air filtration means;
said sealing comprising a substantially air-tight border comprising heat seal, adhesive, fastener or similar joining means.

21. The surgical sheath as recited in claim 12, wherein said surgical sheath is manufactured from impermeable flexible polymer, such as polyethylene, low-density polyethylene (LDPE) polypropylene. poly(tetrafluoroethylene) or silicones.

22. The surgical sheath as recited in claim 12, wherein an extension of said surgical sheath is manufactured from substantially rigid polymer, such as high-density polyethylene (HDPE) polypropylene (PP), polystyrene (PS), Nylon, thermoplastic polyurethanes (TPU), or acrylics.

23. A method for preventing contamination of a sterile field in a surgical operating room when using a medical vacuum device having a vacuum tube, said method comprising the steps of:
placing a substantially flexible tubular sheath over said vacuum tube used in the surgical operating room, said vacuum tube comprising a distal end;
coupling an integral sterile substantially rigid extension to an inlet end of said medical vacuum device;
placing a substantially tubular sheath over at least a portion of said vacuum tube, said substantially tubular sheath comprising a filtration area having a filter;
said substantially tubular sheath being adapted and sized to receive at least a portion of said vacuum tube, thereby creating a sterile transition zone over at least one of a length of said substantially rigid extension or said at least a portion of said vacuum tube.

24. The method as recited in claim 23, wherein said substantially rigid extension comprises an air-impermeable body adapted to be mounted on said vacuum end of said vacuum tube and said substantially tubular sheath comprises a non-permeable sheath portion for facilitating preventing contamination of an external surface of said vacuum tube.

25. A surgical drape system for use with a vacuum tube in a surgical environment, said surgical drape system comprising:
a first portion for coupling to an inlet end of said vacuum tube, said first portion having a first portion inlet end;
a drape portion coupled to or integral with said first portion for situating about at least a portion of said vacuum tube; and
said first portion having an outlet end coupled to said inlet end of said vacuum tube and said drape portion being situated about said at least a portion of said vacuum tube to facilitate preventing contamination from said vacuum tube from spreading into said surgical environment.

26. The surgical drape system as recited in claim 25, wherein said drape is secured to said first portion with a substantially air-tight seal defined by at least one of a weld, adhesive, elastic band, tie, press fit, strap, hook and tie, threads or the like.

27. The surgical drape system as recited in claim 25, wherein said first portion defines an extender or spacer for separating a distance between said inlet end of said vacuum tube and said first portion inlet end for receiving contaminated air, said drape facilitating preventing said contamination from contaminating said vacuum tube.

28. The surgical drape system as recited in claim 25, wherein said drape is coupled or secured to an end of said first portion and extends over said vacuum tube.

29. The surgical drape system as recited in claim 25, wherein said drape is coupled or secured to an end of said first portion and extends over said vacuum tube and at least a portion of said first portion.

30. The surgical drape system as recited in claim 25, wherein said drape and said first portion are impermeable to air; said surgical drape system having at least one filter situated in said first portion inlet end to permit contaminated air to enter said first portion and then said vacuum tube.

31. The surgical drape system as recited in claim 30, wherein said first portion inlet end comprises a predetermined shape, said at least one filter being sized and adapted to complement a shape.

32. The surgical drape system as recited in claim 31, wherein said predetermined shape is a frusto-conical shape.

33. The surgical drape system as recited in claim 25, wherein said outlet end of said first portion is sized and adapted to be received in said inlet end of said vacuum tube so that said drape drapes over an outer surface of said vacuum tube to facilitate preventing contamination from transferring from said vacuum tube to said surgical environment, said outlet end being secured to said inlet end of said vacuum tube with a substantially air-tight seal.

34. The surgical drape system as recited in claim 25, wherein said inlet end of said vacuum tube is sized and adapted to receive said outlet end of said first portion so that said drape drapes over an outer surface of said vacuum tube to facilitate preventing contamination from transferring from said vacuum tube to said surgical environment, said inlet end of said vacuum tube being secured to said outlet end with a substantially air-tight seal.

35. The surgical drape system as recited in claim 25, wherein said outlet end is sealingly coupled to said vacuum tube so that said drape drapes over an outer surface of said vacuum tube to facilitates preventing contamination from transferring from said outer surface of said vacuum tube to said surgical environment

36. The surgical drape system as recited in claim 25, wherein said inlet end of said first portion is frusto-conical in cross section, said first portion having a complementary shape so that it fits inside said inlet end of said vacuum tube, said first portion having a filter that is also frusto-conical in shape and adapted to be housed in said inlet end of said first portion.

37. The surgical drape system as recited in claim 36, wherein said drape is integral with or secured to an inlet end of said first portion, said outlet end of said first portion being situated in proximity to said inlet end of said vacuum tube after said first portion is mounted thereon, said drape being adapted to be situated over both said first portion and at least a portion of said vacuum tube.

38. The surgical drape system as recited in claim 25, wherein said surgical drape system further comprises:
at least one of an air handler or HVAC system coupled to an inlet end of said vacuum tube for creating a negative pressure or vacuum in said vacuum tube in order to vacuum air into an inlet end of said first portion after it is mounted on said vacuum tube.
